(19) United States
(12) Patent Application Publication
(10) Pub. No.: US 2024/0041875 A1
(43) Pub. Date: Feb. 8, 2024

(54) METHODS OF TREATING A CORONAVIRUS INFECTION

(71) Applicant: City of Hope, Duarte, CA (US)

(72) Inventors: Jianhua Yu, Arcadia, CA (US); Michael A. Caligiuri, Pasadena, CA (US); Lei Tian, Duarte, CA (US)

(21) Appl. No.: 18/361,492

(22) Filed: Jul. 28, 2023

Related U.S. Application Data
(60) Provisional application No. 63/393,680, filed on Jul. 29, 2022.

(51) Int. Cl. A61K 31/5025; A61K 39/42; A61K 31/427; A61K 31/4025; A61K 31/675; A61P 31/14

(52) U.S. Cl. CPC A61K 31/5025; A61K 39/42; A61K 31/427; A61K 31/4025; A61K 31/675; A61P 31/14

(57) ABSTRACT

Described herein, inter alia, are methods of treating a coronavirus infection.

### CROSS-REFERENCES TO RELATED APPLICATIONS

This application claims the benefit of U.S. Provisional Application No. 63/393,680, filed Jul. 29, 2022, which is incorporated herein by reference in its entirety and for all purposes.

### BACKGROUND

SARS-CoV-2 is the pathogen responsible for the global COVID-19 pandemic (1). To date, nearly 425,000,000 cases and 6,000,000 deaths have been recorded, with a worldwide mortality rate of 2% (2). Not surprisingly, the public health and economic consequences have been devastating. Although some strategies such as FDA-approved vaccines and some drugs have been effective in the clinic thus far, the pandemic rages on, resulting in persistent concerns regarding the emergence of new variants (3-5). Novel, effective, and safe drugs, especially those that can be orally administered, are urgent needed to halt the pandemic and to save the lives of COVID-19 patients with severe conditions. Disclosed herein, inter alia, are solutions to these and other problems in the art.

### BRIEF SUMMARY

In an aspect is provided a method of treating a coronavirus infection in a subject in need thereof, the method including administering to the subject a therapeutically effective amount of ponatinib.

### BRIEF DESCRIPTION OF THE DRAWINGS

FIG. 1. Screening FDA-approved clinical drugs in a library containing 147 drugs for

inhibiting infection by VSV-SARS-CoV-2. 2×10⁴ Vero cells were seeded in a flat 96-well plate overnight. On the second day, when the cell confluence reached ˜70%, each drug in the library with the concentration of 5 μM was added and incubated at 37° C. for 2 hr, and then VSV-SARS-CoV-2 was added at a multiplicity of infection (MOI) of 0.01. One hour later, the drugs-virus mixture was removed, and the infected cells were washed twice with pre-warmed PBS, and then 10% FBS DMEM medium was added. 48 hr later, fluorescence microscopy was used to examine infected cells, marked by GFP expression. Data for a part of drugs in the library on one 96-well plate are shown. A06: Ponatinib; A08: Dacomitinib; B05: Neratinib.

FIG. 2. Confirming screened drugs in Vero cells. 2×10⁴ Vero cells were seeded in a flat 96-well plate overnight. On the second day, when the cell confluence reached ˜70%, each indicated drug with indicated concentration was added and incubated at 37° C. for 2 hr, and then VSV-SARS-CoV-2 was added at a MOI of 0.01. One hour later, the drugs-virus mixture was removed, and the infected cells were washed twice with pre-warmed PBS, and then 10% FBS DMEM medium was added. 48 hr later, fluorescence microscopy was used to examine infected cells, marked by GFP expression.

FIG. 3. Confirming screened ponatinib in A549 cells ectopically expressing ACE2 (A549-ACE2). 4×10⁴ A549-ACE2 cells were seeded in a flat 96-well plate overnight. On the second day, when the cell confluence reached ˜70%, VSV-SARS-CoV-2 was preincubated with ponatinib at different concentrations as indicated for 1 hour and washed. The pre-incubated viruses after the wash were used to infect A549-ACE2 cells at a multiplicity of infection (MOI) of 0.2. The infected cells were imaged at 48 hours post infection (hpi) by fluorescence microscopy.

FIGS. 4A-4B. Inhibition of authentic SARS-CoV-2 infection in A549-ACE2 cells. 4×10⁵ A549-ACE2 cells were seeded in a flat 96-well plate overnight. On the second day, when the cell confluence reached ˜70%, A549-ACE2 cells were either pre-incubated with 5 nM or 10 nM ponatinib for 1 hour and washed. The pre-incubated viruses after the wash were used to infect A549-ACE2 cells at a multiplicity of infection (MOI) of 0.2. The infected cells were imaged at 48 hours post infection (hpi) by fluorescence microscopy.

FIG. 5. Ponatinib acts on both virus and host cells. VSV-SARS-CoV-2 and ponatinib (2.5 nM) were treated at indicated conditions (1-4). A549-ACE2 cells were infected at a MOI of 0.2 and imaged at 48 hpi by fluorescence microscopy. Conditions: 1. Cells were concomitantly treated with VSV-SARS-CoV-2 and ponatinib for 1 hr and then washed. 2.VSV-SARS-CoV-2 was co-incubated with ponatinib 1 hr, and washed and then added to cells. 3. Cells were pre-incubated with ponatinib, washed, and then VSV-SARS-CoV-2 was added. 4. Cells were infected with VSV-SARS-CoV-2 for 1 hr, and then VSV-SARS-CoV-2 was added.

FIG. 6. Ponatinib cytotoxicity to A549-ACE2 cells at the concentration range of 0.1625 nM to 2.5 nM). A549-ACE2 cells were treated with ponatinib for 24 h and the cell viability was detected by CellTiter-Glo Assay.

FIGS. 7A-7B. Body weights (FIG. 7A) and survival (FIG. 7B) of mice infected with 5×10³ PFU SARS-CoV-2 WT strain and treated with or without 3 mg/kg ponatinib by oral gavage once daily. Body weights of each mouse were monitored. PBS alone was used as control. n=4 mice for PBS. n=5 mice for ponatinib group.

FIG. 8. Viral RNA copies in the lungs of infected mice that were treated daily with or without 3 mg/kg ponatinib were assessed by Q-PCR. Mice were infected with 5×10³ PFU SARS-CoV-2 WT strain for 4 days prior to being sacrificed to harvest lungs to measure the viral titer. PBS alone serves as the control.

FIG. 9. SARS-CoV-2 viral titers were detected in the lungs of infected mice that were daily treated with 3 mg/kg ponatinib or PBS by plaque assays. Mice were infected with 5×10³ PFU SARS-CoV-2 WT strain for 4 days prior to being sacrificed to harvest lungs to measure the viral titer. PBS alone serves as control.

FIG. 10. SARS-CoV-2 was detected in the lung of mice daily treated with 3 mg/kg ponatinib or PBS by using IHC staining with an antibody against SARS-CoV-2 nucleocapsid protein (NP). Mice were infected with 5×10³ PFU SARS-CoV-2 WT strain for 4 days prior to being sacrificed to heaviest lungs to measure the viral titer. PBS alone serves as control.

FIG. 11. A549-ACE2 cells were concomitantly VSV-SARS-CoV-2 at a MOI of 0.1 and ponatinib or olverembatinib at indicated concentrations and imaged at 48 hpi by fluorescence microscopy.

FIG. 12. Dose-dependent effect. Survival of mice infected with 5×10³ PFU SARS-CoV-2 WT strain and treated indicated doses of ponatinib by oral gavage once daily. n=3 mice for 0.75 mg/kg group; n=5 mice for 1.5 mg/kg and 3 mg/kg ponatinib groups.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

In this disclosure, “comprises”, “comprising”, “containing”, and “having” and the like can have the meaning ascribed to them in U.S. Patent law and can mean “ includes”, “including”, and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments, inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g., a protein associated disease, disease associated with a cellular component) means that the disease (e.g., coronavirus infection) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

“Disease” or “condition” refers to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a coronavirus infection. In embodiments, the disease is a SARS-CoV infection. In embodiments, the disease is a SARS-CoV-2 infection. In embodiments, the disease is coronavirus disease 2019 (COVID-19).

The term “coronavirus” is used in accordance with its plain ordinary meaning and refers to an RNA virus that in humans causes respiratory tract infections. Coronaviruses constitute the subfamily Orthocoronavirinae, in the family Coronaviridae, order Nidovirales, and realm Riboviria. In embodiments, the coronavirus is an enveloped viruses with a positive-sense single-stranded RNA genome.

The term “severe acute respiratory syndrome coronavirus” or “SARS-CoV” or “SARS-CoV-1” refers to the strain of coronavirus that causes severe acute respiratory syndrome (SARS). In embodiments, SARS-CoV-1 is an enveloped, positive-sense, single-stranded RNA virus that infects the epithelial cells within the lungs. In embodiments, the virus enters the host cell by binding to the angiotensin-converting enzyme 2 (ACE2) receptor.

The term “severe acute respiratory syndrome coronavirus 2” or “SARS-CoV-2” refers to the strain of coronavirus that causes coronavirus disease 2019 (COVID-19). In embodiments, SARS-CoV-2 is a positive-sense single-stranded RNA virus.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, the term “SARS-CoV-2 protein” refers to a protein of the SARS-CoV-2 virus.

The term “coronavirus spike protein” is used in accordance with its plain and ordinary meaning in the art and refers to a protein found in coronaviruses. The coronavirus spike protein mediates viral entry into the host cell. In embodiments, the coronavirus spike protein has the amino acid sequence set forth in or corresponding to UniProt P0DTC2, or homolog thereof. In embodiments, the amino acid sequence or nucleic acid sequence is the sequence known at the time of filing of the present application.

The term “ACE2” or “angiotensin-converting enzyme 2” refers to a protein encoded by the ACE2 gene. The term includes any recombinant or naturally-occurring form of ACE2 variants thereof that maintain ACE2 activity (e.g., within at least 30%, 40%, 50%, 60%, 70%, 80%, 90%, 95%, or 100% activity compared to wildtype ACE2). In embodiments, the ACE2 protein encoded by the ACE2 gene has the amino acid sequence set forth in or corresponding to Entrez 59272, UniProt Q9BYF1, RefSeq (protein) NP_068576.1, or RefSeq (protein) NP_001358344, or homolog thereof. In embodiments, the amino acid sequence or nucleic acid sequence is the sequence known at the time of filing of the present application.

The term “ACE2 fusion protein” is used in accordance with its plain and ordinary meaning in the art and refers to a protein (e.g., coronavirus protein) that interacts with ACE2, the host cell receptor to which SARS-CoV-2 binds, in order to initiate viral entry. In embodiments, the coronavirus spike protein has the amino acid sequence set forth in or corresponding to UniProt P0DTC2, or homolog thereof. In embodiments, the amino acid sequence or nucleic acid sequence is the sequence known at the time of filing of the present application.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat a coronavirus infection by decreasing the incidence of the coronavirus infection and or causing remission of the coronavirus infection. The term “treating” and conjugations thereof, include prevention of an injury, pathology, condition, or disease. In embodiments, treating is preventing. In embodiments, treating does not include preventing. In embodiments, the treating or treatment is not prophylactic treatment.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

As used herein, a “symptom” of a disease includes any clinical or laboratory manifestation associated with the disease, and is not limited to what a subject can feel or observe.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goats, sheep, cows, deer, and other non-mammalian animals. In some embodiments, a patient is human. In embodiments, a patient in need thereof is human. In embodiments, a subject is human. In embodiments, a subject in need thereof is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compound's effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

In therapeutic use for the treatment of a disease, a compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., coronavirus infection) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

“Co-administer” is meant that a composition described herein is administered at the same time, just prior to, or just after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example, mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

The terms “bind” and “bound” as used herein are used in accordance with their plain and ordinary meaning and refer to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der Waals bond(s)/interactions, hydrogen bond(s), polar bond(s), or combinations or mixtures thereof).

The term “drug” is used in accordance with its plain and ordinary meaning and refers to a substance that has a physiological effect (e.g., beneficial effect, is useful for treating a subject) when introduced into or to a subject (e.g., in or on the body of a subject or patient). A drug moiety is a radical of a drug.

The term “antiviral agent” is used in accordance with its plain and ordinary meaning and refers to a compound or composition useful in treating a viral infection.

The term “coronavirus therapeutic agent” as used herein refers to a compound or composition useful in treating a coronavirus infection.

### II. Methods

In an aspect is provided a method of treating a coronavirus infection in a subject in need thereof, the method including administering to the subject a therapeutically effective amount of ponatinib.

In an aspect is provided a method of treating a coronavirus infection in a subject in need thereof, the method including administering to the subject a therapeutically effective amount of dacomitinib.

In an aspect is provided a method of treating a coronavirus infection in a subject in need thereof, the method including administering to the subject a therapeutically effective amount of neratinib.

In an aspect is provided a method of treating a coronavirus infection in a subject in need thereof, the method including administering to the subject a therapeutically effective amount of ponatinib, or an analog thereof. In embodiments, the analog of ponatinib is as described in U.S. Pat. Nos. 8,114,874; 9,029,533; 9,493,470; 11,192,895; or 11,192,897; which are incorporated herein by reference in their entirety and for all purposes.

In embodiments, ponatinib is

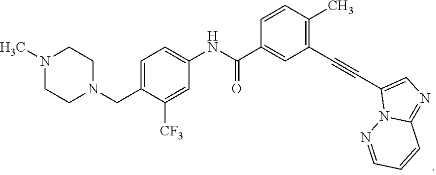 [SMILES: Cc1ccc(C(=O)Nc2ccc(CN3CCN(C)CC3)c(C(F)(F)F)c2)cc1C#Cc1cnc2cccnn12]

In an aspect is provided a method of treating a coronavirus infection in a subject in need thereof, the method including administering to the subject a therapeutically effective amount of dacomitinib, or an analog thereof. In embodiments, the analog of dacomitinib is as described in U.S. Pat. Nos. 7,772,243; 8,623,883; 10,596,162; or 10,603,314; which are incorporated herein by reference in their entirety and for all purposes.

In embodiments, dacomitinib is

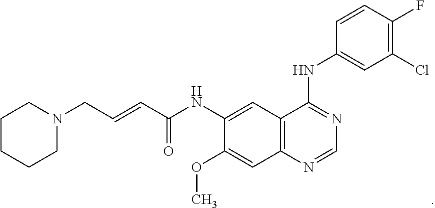 [SMILES: COc1cc2ncnc(Nc3ccc(F)c(Cl)c3)c2cc1NC(=O)/C=C/CN1CCCCC1]

In an aspect is provided a method of treating a coronavirus infection in a subject in need thereof, the method including administering to the subject a therapeutically effective amount of neratinib, or an analog thereof. In embodiments, the analog of neratinib is as described in U.S. Pat. Nos. 7,399,865; 7,982,043; 8,518,446; 8,669,273; 8,790,708; 9,139,558; 9,211,291; 9,265,784; 9,630,946; or 10,035,788; which are incorporated herein by reference in their entirety and for all purposes.

In embodiments, neratinib is

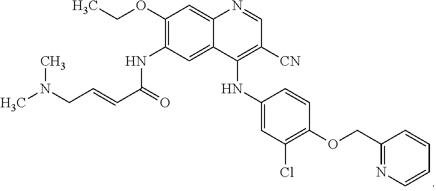 [SMILES: CCOc1cc2ncc(C#N)c(Nc3ccc(OCc4ccccn4)c(Cl)c3)c2cc1NC(=O)/C=C/CN(C)C]

In embodiments, the coronavirus infection is a SARS-CoV infection. In embodiments, the coronavirus infection is Severe Acute Respiratory Disease (SARS). In embodiments, the coronavirus infection is a SARS-CoV-2 infection. In embodiments, the coronavirus infection is coronavirus disease 2019 (COVID-19). In embodiments, the subject in need thereof has or is suspected of having COVID-19. In embodiments, the coronavirus infection is a MERS-CoV infection. In embodiments, the coronavirus infection is an HCoV-NL63 infection. In embodiments, the coronavirus infection is an HCoV-229E infection. In embodiments, the coronavirus infection is an HCoV-OC43 infection. In embodiments, the coronavirus infection is an HKU1 infection.

In embodiments, the therapeutically effective amount is from 0.1 mg per day to 60 mg per day. In embodiments, the therapeutically effective amount is from 0.1 mg per day to 30 mg per day. In embodiments, the therapeutically effective amount is from 0.1 mg per day to 15 mg per day. In embodiments, the therapeutically effective amount is from 1 mg per day to 15 mg per day. In embodiments, the therapeutically effective amount is from 1 mg per day to 10 mg per day. In embodiments, the therapeutically effective amount is less than 15 mg per day. In embodiments, the therapeutically effective amount is 0.1 mg per day. In embodiments, the therapeutically effective amount is 0.2 mg per day. In embodiments, the therapeutically effective amount is 0.3 mg per day. In embodiments, the therapeutically effective amount is 0.4 mg per day. In embodiments, the therapeutically effective amount is 0.5 mg per day. In embodiments, the therapeutically effective amount is 0.6 mg per day. In embodiments, the therapeutically effective amount is 0.7 mg per day. In embodiments, the therapeutically effective amount is 0.8 mg per day. In embodiments, the therapeutically effective amount is 0.9 mg per day. In embodiments, the therapeutically effective amount is 1 mg per day. In embodiments, the therapeutically effective amount is 2 mg per day. In embodiments, the therapeutically effective amount is 3 mg per day. In embodiments, the therapeutically effective amount is 4 mg per day. In embodiments, the therapeutically effective amount is 5 mg per day. In embodiments, the therapeutically effective amount is 6 mg per day. In embodiments, the therapeutically effective amount is 7 mg per day. In embodiments, the therapeutically effective amount is 8 mg per day. In embodiments, the therapeutically effective amount is 9 mg per day. In embodiments, the therapeutically effective amount is 10 mg per day. In embodiments, the therapeutically effective amount is 11 mg per day. In embodiments, the therapeutically effective amount is 12 mg per day. In embodiments, the therapeutically effective amount is 13 mg per day. In embodiments, the therapeutically effective amount is 14 mg per day. In embodiments, the therapeutically effective amount is 15 mg per day. In embodiments, the therapeutically effective amount is 16 mg per day. In embodiments, the therapeutically effective amount is 17 mg per day. In embodiments, the therapeutically effective amount is 18 mg per day. In embodiments, the therapeutically effective amount is 19 mg per day. In embodiments, the therapeutically effective amount is 20 mg per day. In embodiments, the therapeutically effective amount is 21 mg per day. In embodiments, the therapeutically effective amount is 22 mg per day. In embodiments, the therapeutically effective amount is 23 mg per day. In embodiments, the therapeutically effective amount is 24 mg per day. In embodiments, the therapeutically effective amount is 25 mg per day. In embodiments, the therapeutically effective amount is 26 mg per day. In embodiments, the therapeutically effective amount is 27 mg per day. In embodiments, the therapeutically effective amount is 28 mg per day. In embodiments, the therapeutically effective amount is 29 mg per day. In embodiments, the therapeutically effective amount is 30 mg per day.

In embodiments, the therapeutically effective amount is from about 0.1 mg per day to about 60 mg per day. In embodiments, the therapeutically effective amount is from about 0.1 mg per day to about 30 mg per day. In embodiments, the therapeutically effective amount is from about 0.1 mg per day to about 15 mg per day. In embodiments, the therapeutically effective amount is from about 1 mg per day to about 15 mg per day. In embodiments, the therapeutically effective amount is from about 1 mg per day to about 10 mg per day. In embodiments, the therapeutically effective amount is less than about 15 mg per day. In embodiments, the therapeutically effective amount is about 0.1 mg per day. In embodiments, the therapeutically effective amount is about 0.2 mg per day. In embodiments, the therapeutically effective amount is about 0.3 mg per day. In embodiments, the therapeutically effective amount is about 0.4 mg per day. In embodiments, the therapeutically effective amount is about 0.5 mg per day. In embodiments, the therapeutically effective amount is about 0.6 mg per day. In embodiments, the therapeutically effective amount is about 0.7 mg per day. In embodiments, the therapeutically effective amount is about 0.8 mg per day. In embodiments, the therapeutically effective amount is about 0.9 mg per day. In embodiments, the therapeutically effective amount is about 1 mg per day. In embodiments, the therapeutically effective amount is about 2 mg per day. In embodiments, the therapeutically effective amount is about 3 mg per day. In embodiments, the therapeutically effective amount is about 4 mg per day. In embodiments, the therapeutically effective amount is about 5 mg per day. In embodiments, the therapeutically effective amount is about 6 mg per day. In embodiments, the therapeutically effective amount is about 7 mg per day. In embodiments, the therapeutically effective amount is about 8 mg per day. In embodiments, the therapeutically effective amount is about 9 mg per day. In embodiments, the therapeutically effective amount is about 10 mg per day. In embodiments, the therapeutically effective amount is about 11 mg per day. In embodiments, the therapeutically effective amount is about 12 mg per day. In embodiments, the therapeutically effective amount is about 13 mg per day. In embodiments, the therapeutically effective amount is about 14 mg per day. In embodiments, the therapeutically effective amount is about 15 mg per day. In embodiments, the therapeutically effective amount is about 16 mg per day. In embodiments, the therapeutically effective amount is about 17 mg per day. In embodiments, the therapeutically effective amount is about 18 mg per day. In embodiments, the therapeutically effective amount is about 19 mg per day. In embodiments, the therapeutically effective amount is about 20 mg per day. In embodiments, the therapeutically effective amount is about 21 mg per day. In embodiments, the therapeutically effective amount is about 22 mg per day. In embodiments, the therapeutically effective amount is about 23 mg per day. In embodiments, the therapeutically effective amount is about 24 mg per day. In embodiments, the therapeutically effective amount is about 25 mg per day. In embodiments, the therapeutically effective amount is about 26 mg per day. In embodiments, the therapeutically effective amount is about 27 mg per day. In embodiments, the therapeutically effective amount is about 28 mg per day. In embodiments, the therapeutically effective amount is about 29 mg per day. In embodiments, the therapeutically effective amount is about 30 mg per day.

In embodiments, ponatinib is administered daily from 1 to 21 days. In embodiments, ponatinib is administered daily from 1 to 14 days (e.g., after infection). In embodiments, ponatinib is administered daily from 1 to 10 days (e.g., after infection). In embodiments, ponatinib is administered daily from 1 to 9 days (e.g., after infection). In embodiments, ponatinib is administered daily from 1 to 8 days (e.g., after infection). In embodiments, ponatinib is administered daily from 1 to 7 days (e.g., after infection). In embodiments, ponatinib is administered daily from 1 to 6 days (e.g., after infection). In embodiments, ponatinib is administered daily from 1 to 5 days (e.g., after infection). In embodiments, ponatinib is administered daily from 3 to 10 days (e.g., after infection). In embodiments, ponatinib is administered daily from 3 to 9 days (e.g., after infection). In embodiments, ponatinib is administered daily from 3 to 8 days (e.g., after infection). In embodiments, ponatinib is administered daily from 3 to 7 days (e.g., after infection). In embodiments, ponatinib is administered daily from 3 to 6 days after infection (e.g., after infection). In embodiments, ponatinib is administered daily from 3 to 5 days (e.g., after infection).

In embodiments, ponatinib is administered daily for 1 day (e.g., after infection). In embodiments, ponatinib is administered daily for 2 days (e.g., after infection). In embodiments, ponatinib is administered daily for 3 days (e.g., after infection). In embodiments, ponatinib is administered daily for 4 days (e.g., after infection). In embodiments, ponatinib is administered daily for 5 days (e.g., after infection). In embodiments, ponatinib is administered daily for 6 days (e.g., after infection). In embodiments, ponatinib is administered daily for 7 days (e.g., after infection). In embodiments, ponatinib is administered daily for 8 days (e.g., after infection). In embodiments, ponatinib is administered daily for 9 days (e.g., after infection). In embodiments, ponatinib is administered daily for 10 days (e.g., after infection). In embodiments, ponatinib is administered daily for 11 days (e.g., after infection). In embodiments, ponatinib is administered daily for 12 days (e.g., after infection). In embodiments, ponatinib is administered daily for 13 days (e.g., after infection). In embodiments, ponatinib is administered daily for 14 days (e.g., after infection). In embodiments, ponatinib is administered daily for 15 days (e.g., after infection). In embodiments, ponatinib is administered daily for 16 days (e.g., after infection). In embodiments, ponatinib is administered daily for 17 days (e.g., after infection). In embodiments, ponatinib is administered daily for 18 days (e.g., after infection). In embodiments, ponatinib is administered daily for 19 days (e.g., after infection). In embodiments, ponatinib is administered daily for 20 days (e.g., after infection). In embodiments, ponatinib is administered daily for 21 days (e.g., after infection).

In embodiments, ponatinib is administered daily from 1 to 21 days after infection. In embodiments, the date of infection is the first day symptoms are observed. In embodiments, the symptoms include, but are not limited to, fever, chills, cough, shortness of breath or difficulty breathing, fatigue, muscle pain, body aches, headache, new loss of taste or smell, sore throat, congestion or runny nose, nausea or vomiting, and diarrhea. In embodiments, the date of infection is the date of a first positive coronavirus infection test.

In embodiments, ponatinib is administered orally. In embodiments, ponatinib is administered intravenously. In embodiments, ponatinib is administered intramuscularly.

In embodiments, the method further includes administering to the subject a therapeutically effective amount of an antiviral agent in addition to ponatinib. In embodiments, the method further includes administering to the subject a therapeutically effective amount of an antiviral agent in addition to dacomitinib. In embodiments, the method further includes administering to the subject a therapeutically effective amount of an antiviral agent in addition to neratinib.

In embodiments, the antiviral agent is a coronavirus therapeutic agent. In embodiments, coronavirus therapeutic agent is an antibody against a SARS-CoV-2 protein. In embodiments, the SARS-CoV-2-protein is a spike protein. In embodiments, the SARS-CoV-2-protein is an ACE2 fusion protein. In embodiments, the coronavirus therapeutic agent is Paxlovid or Veklury. In embodiments, the coronavirus therapeutic agent is nirmatrelvir. In embodiments, the coronavirus therapeutic agent is ritonavir. In embodiments, the coronavirus therapeutic agent is a combination of nirmatrelvir and ritonavir (Paxlovid). In embodiments, the coronavirus therapeutic agent is remdesivir (Veklury).

In embodiments, the method does not include administering to the subject a therapeutically effective amount of an antiviral agent.

### III. Pharmaceutical Compositions

In an aspect is provided a pharmaceutical composition including ponatinib, or a pharmaceutically acceptable salt thereof, and a pharmaceutically acceptable excipient.

In embodiments, the pharmaceutical composition includes an effective amount of ponatinib. In embodiments, the pharmaceutical composition includes a therapeutically effective amount of ponatinib.

In embodiments, the pharmaceutical composition further includes an antiviral agent (e.g., as described herein).

In embodiments, the pharmaceutical composition does not include an antiviral agent.

### IV. Embodiments

Embodiment P1. A method of treating a coronavirus infection in a subject in need thereof, said method comprising administering to the subject a therapeutically effective amount of ponatinib.

Embodiment P2. The method of embodiment P1, wherein the coronavirus infection is a SARS-CoV-2 infection.

Embodiment P3. The method of one of embodiments P1 to P2, wherein the therapeutically effective amount is from 0.1 mg per day to 30 mg per day.

Embodiment P4. The method of one of embodiments P1 to P2, wherein the therapeutically effective amount is from 0.1 mg per day to 15 mg per day.

Embodiment P5. The method of one of embodiments P1 to P2, wherein the therapeutically effective amount is from 1 mg per day to 15 mg per day.

Embodiment P6. The method of one of embodiments P1 to P2, wherein the therapeutically effective amount is from 1 mg per day to 10 mg per day.

Embodiment P7. The method of one of embodiments P1 to P2, wherein the therapeutically effective amount is less than 15 mg per day.

Embodiment P8. The method of one of embodiments P1 to P7, wherein ponatinib is administered daily from 1 to 21 days.

Embodiment P9. The method of one of embodiments P1 to P7, wherein ponatinib is administered daily from 1 to 14 days.

Embodiment P10. The method of one of embodiments P1 to P7, wherein ponatinib is administered daily from 1 to 10 days.

Embodiment P11. The method of one of embodiments P1 to P7, wherein ponatinib is administered daily from 1 to 7 days.

Embodiment P12. The method of one of embodiments P1 to P7, wherein ponatinib is administered daily from 3 to 10 days.

Embodiment P13. The method of one of embodiments P1 to P7, wherein ponatinib is administered daily from 3 to 7 days.

Embodiment P14. The method of one of embodiments P1 to P13, wherein ponatinib is administered orally.

Embodiment P15. The method of one of embodiments P1 to P13, wherein ponatinib is administered intravenously.

Embodiment P16. The method of one of embodiments P1 to P13, wherein ponatinib is administered intramuscularly.

Embodiment P17. The method of one of embodiments P1 to P16, further comprising administering to the subject a therapeutically effective amount of a second agent.

Embodiment P18. The method of embodiment P17, wherein the second agent is an antibody against a SARS-CoV-2 protein.

Embodiment P19. The method of embodiment P18, wherein the SARS-CoV-2-protein is a coronavirus spike protein.

Embodiment P20. The method of embodiment P18, wherein the SARS-CoV-2-protein is an ACE2 fusion protein.

Embodiment P21. The method of embodiment P17, wherein the second agent is Paxlovid or Veklury.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Inhibition of SARS-CoV-2 by Ponatinib

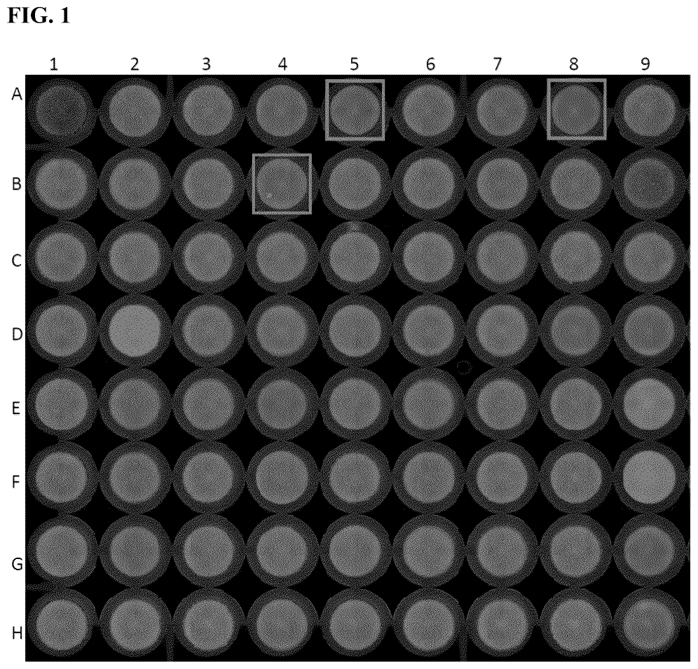

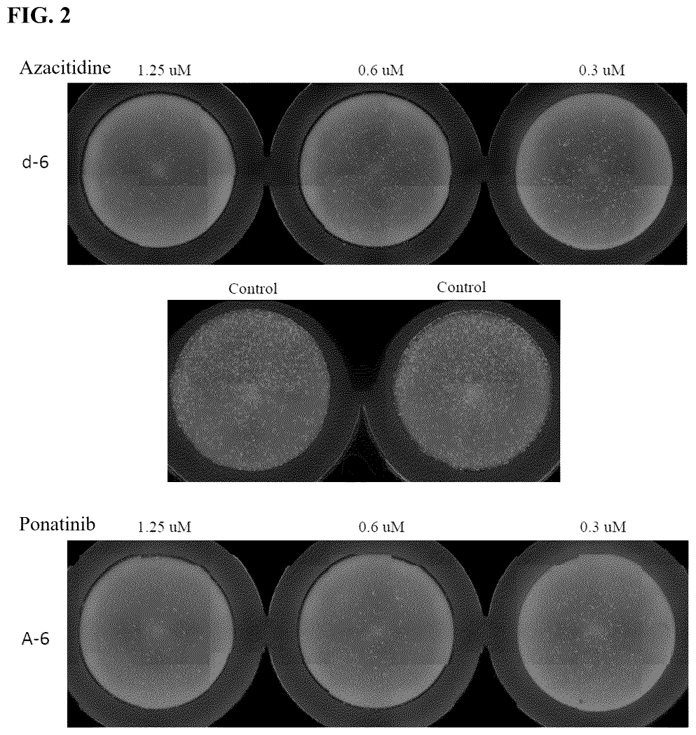

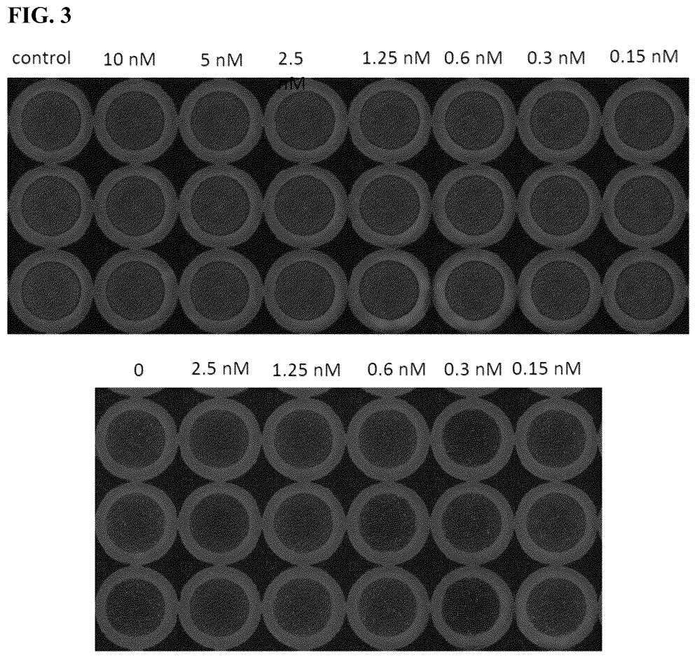

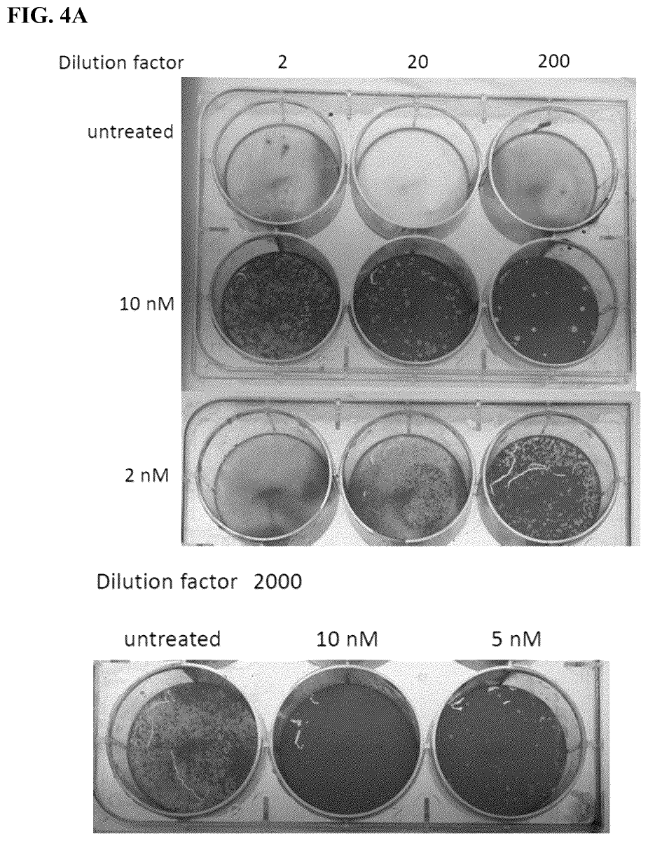

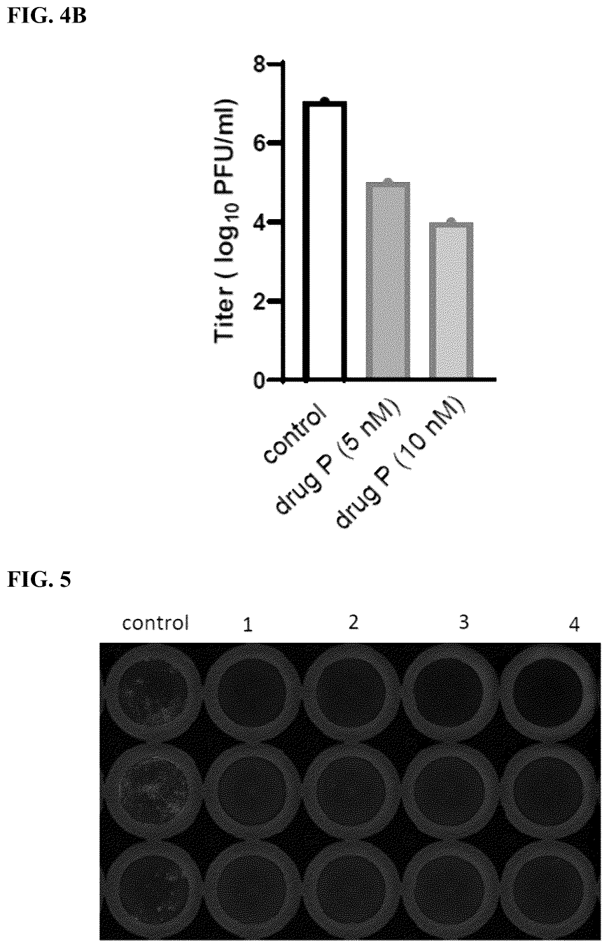

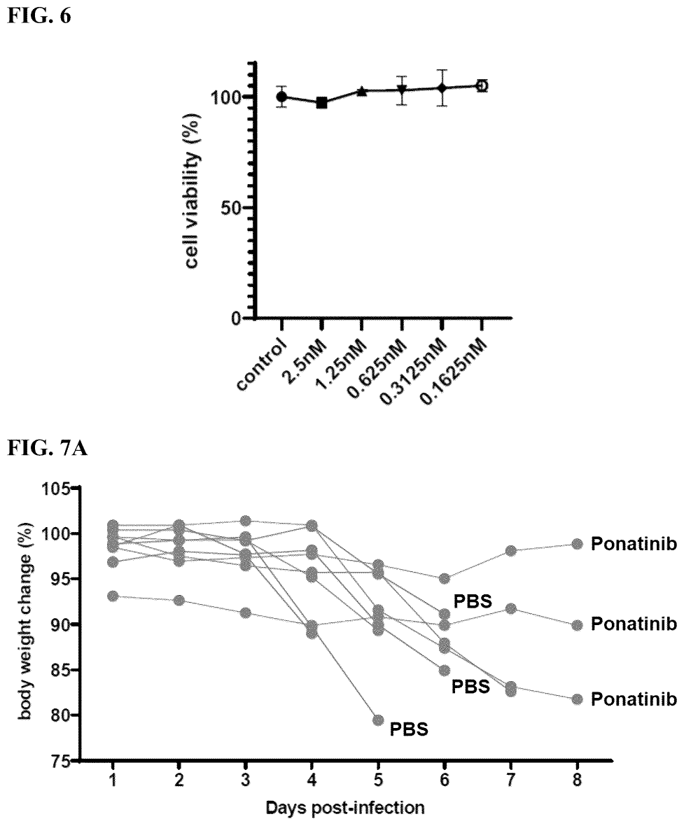

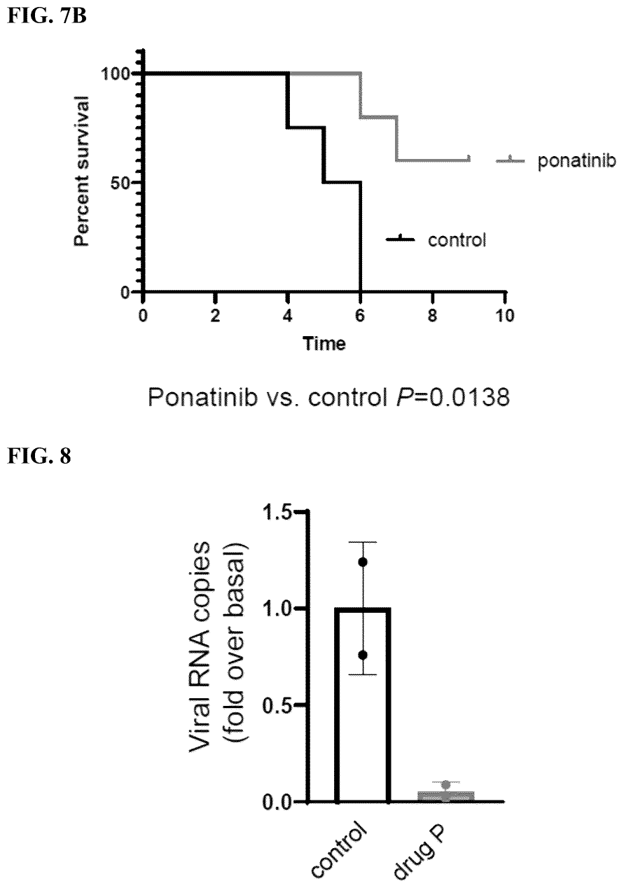

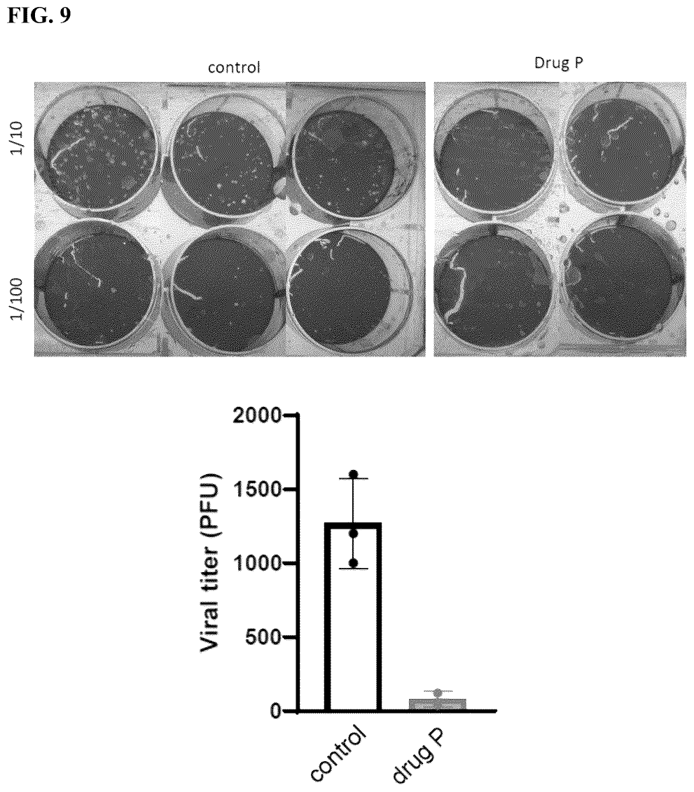

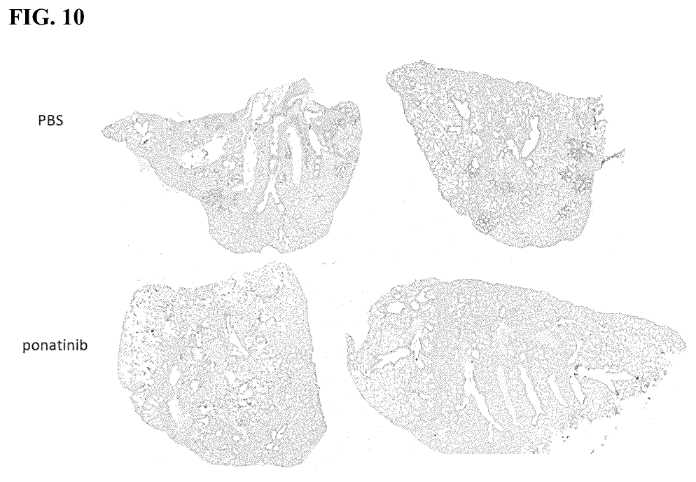

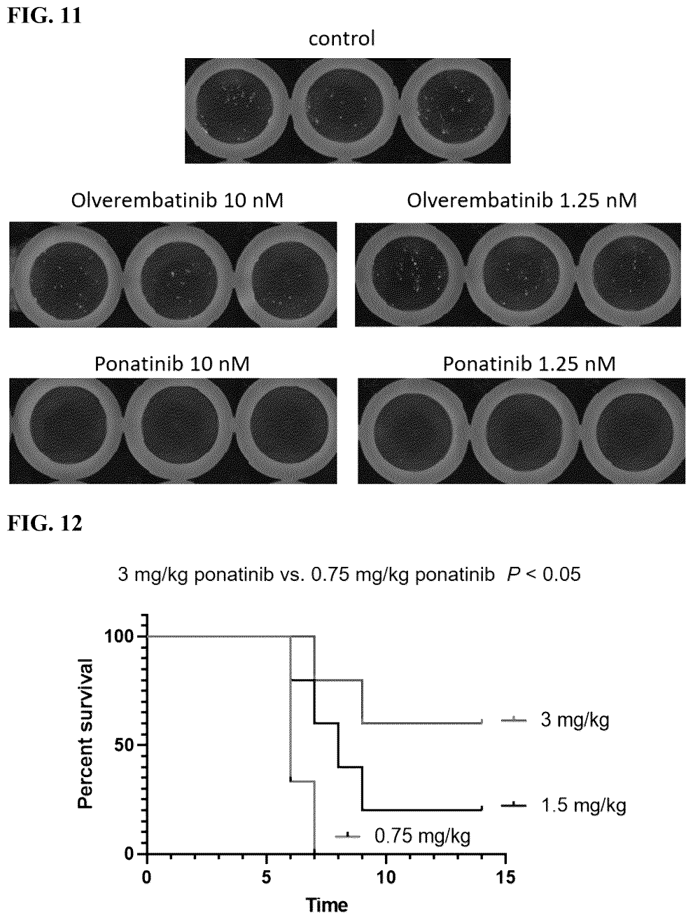

We screened 147 U.S. FDA-approved clinic drugs to examine their potential to inhibit SARS-CoV-2 infection by using the vesicular stomatitis virus (VSV)-SARS-CoV-2 chimeric virus, followed by in vitro and in vivo validation with the authentic SARS-CoV-2 virus. Our screening resulted in several candidates including ponatinib (FIG. 1, FIG. 2). Ponatinib is a third-generation tyrosine kinase inhibitor (TKI) that was originally designed to inhibit the BCR-ABL1 oncogene in chronic myeloid leukemia (CML) (6). FDA approved the drug to treat adults with CML and Philadelphia chromosome positive acute lymphoblastic leukemia (Ph+ALL). New studies indicate that ponatinib also has other targets such as MEKK2 (7), fibroblast growth factor receptor 2 (FGFR2) (8), and others (9).

To validate the role of ponatinib on SARS-CoV-2 infection screened by using the library, we infected A549 lung cells ectopically expressing ACE2 (A549-ACE2) with the VSV-SARS-CoV-2 pre-incubated with different concentrations of ponatinib 1 hour before infection and the infectivity was determined at 48-hour post-infection (hpi). Compared to the control group, ponatinib significantly inhibited VSV-SARS-CoV-2 in a dose-dependent manner (FIG. 3). We next used live SARS-CoV-2 to infect A549-ACE2 cells followed by a quantitative assessment of viral load using a plaque assay. We found that of ponatinib with live SARS-CoV-2 prior to infection significantly reduced viral infection A549-ACE2 cells in a dose-dependent manner compared to the buffer control, consistent with the above data from the chimeric VSV-SARS-CoV-2 virus (FIGS. 4A-4B).

To figure out the potential mechanism of ponatinib in the inhibition of SARS-CoV-2 infection, we first explored the effect of ponatinib on host cells or viruses during the infection. For this purpose, we set up 5 experimental groups: 1) 2.5 nM ponatinib and VSV-SARS-CoV-2 concomitant treatment; 2) VSV-SARS-CoV-2 pre-incubated with 2.5 nM ponatinib 1 hour before infection; 3) A549-ACE2 cells pretreated with 2.5 nM ponatinib 1 hour before VSV-SARS-CoV-2 infection; 4) A549-ACE2 cells infected with VSV-SARS-CoV-2 1 hour prior to 2.5 nM ponatinib treatment and 5) the control group were set up. The results of the infectivity at 48-hour post-infection (hpi) showed that ponatinib could inhibit VSV-SARS-CoV-2 under all these conditions, indicating that ponatinib could target both host and virus to reduce the infectivity (FIG. 5). We also explored whether ponatinib had toxicity on A549-ACE2 cells. It seemed that ponatinib had no effect on A549-ACE2 cell viability (FIG. 6).

To evaluate the potential effect of ponatinib in vivo, we used humanized K18-hACE2 mice as an infection model of SARS-CoV-2. We inoculated those mice with SARS-CoV-2 and then treated 3 mg/kg ponatinib or the PBS control by oral gavage once daily. The body weight of the mice was monitored daily. Most of the mice in the PBS groups exhibited a dramatic decrease in their body weight at day 5 and they were euthanized at that time or shortly thereafter (FIG. 7). In contrast, two of the five mice treated with ponatinib started to regain body weight on day 7 and one's body weight dropped slowly. The ponatinib-treated group also lived significantly longer than the control group (FIGS. 7A-7B). To measure viral copies in the lung, we isolated RNA and used quantitative real-time PCR. The viral copies in the ponatinib-treated group were much lower than in the PBS group, indicating that ponatinib had significantly restricted SARS-CoV-2 infection in vivo (FIG. 8). We also measured the viral titer in the lung by plaque assay and results showed that ponatinib treatment significantly decreased the viral load in the lung (FIG. 9). Consistent with these, IHC showed that expression of the SARS-CoV-2 viral nucleocapsid protein (NP) was also markedly lower in lung tissues in the ponatinib treated group compared to the untreated group (FIG. 10).

Procedure step by step for library screening:
1. On day 0, Vero cells were seeded in a flat-bottom 96-well plate, 1.5×10⁴ cells per well.
2. On day 1, the Vero cells should be in confluency of 70-80% and are ready to use.
   i) Prepare the drugs at 5 μM in medium (10% FBS DMEM).
   ii) Discard the supernatant in Vero cells and wash twice with pre-warmed PBS.
   iii) Add the drugs-containing medium (100 ul/well) to the cells and incubate at 37° C. for 2 hours.
   iv) Two hours later, add the virus (MOI=0.01) into the Vero cells (100 ul/well), and incubate at 37° C. for another 1 hour.
   v) Then, remove the drugs-virus mixture, and wash twice with pre-warmed PBS.
   vi) Add the fresh 10% FBS DMEM medium and incubate another 24 hours to take images.
3. On day 2, take an image.

### Example 2: Comparison Data Between Ponatinib and Olverembatinib

We performed side-by-side comparisons between ponatinib and olverembatinib (FIG. 11). Our data shows that only ponatinib inhibits viral infection. As shown in FIG. 11, the lung cell line A549 expressing ACE2 was infected with VSV-SARS-CoV-2 at a MOI of 0.1 in the presence of two same concentrations (1.25 nM and 10 nM of p) of ponatinib or olverembatinib. The infected cells were detected by GFP expression under a microscope. The data showed that ponatinib but not olverembatinib treatment inhibited plaque formation. Our data confirm that ponatinib inhibits infection of cells by SARS-CoV-2 and also demonstrates the specificity of ponatinib.

### Example 3: Experimental Methods

### Cells

Monkey kidney epithelium-derived Vero cells (Vero E6 cells) and adenocarcinomic human alveolar basal epithelial cells A549 were cultured in DMEM with 10% FBS, penicillin (100 U/ml), and streptomycin (100 μg/ml). All cell lines were routinely tested to confirm the absence of mycoplasma, using the MycoAlert Plus Mycoplasma Detection Kit from Lonza (Walkersville, MD).

### VSV-SARS-CoV-2 Infection

For VSV-SARS-CoV-2 infection, A549-ACE2 cells were seeded 24 hours before the infection at a confluency of 70% in a 96-well plate. VSV-SARS-CoV-2 virus and varying amounts (0.15 nM, 0.3 nM, 0.6 nM, 1.25 nM, 2.5 nM, 5 nM, and 10 nM) of the Ponatinib were co-incubated at 37° C. for 1 hour and then added to the cells. Infectivity was assessed by detecting GFP fluorescence using a Zeiss fluorescence microscope (AXIO observer 7) at 48 hours post infection (hpi).

Screening an FDA-Approved Clinical Drug Library, Containing 147 drugs, for Inhibiting Infection by VSV-SARS-CoV-2

2×10⁴ Vero cells were seeded in a flat 96-well plate overnight. On the second day, when the cell confluence reached ˜70%, each drug in the library with a concentration of 5 μM was added and incubated at 37° C. for 2 hr, and then VSV-SARS-CoV-2 was added at a multiplicity of infection (MOI) of 0.01. One hr later, the drugs-virus mixture was removed, and the infected cells were washed twice with pre-warmed PBS, and then 10% FBS DMEM medium was added. 48 hr later, fluorescence microscopy was used to examine infected cells, marked by GFP expression.

### SARS-CoV-2 Cell Infection and Plaque Assay

The following reagent were obtained through BEI Resources, NIAID, NIH: SARS-Related Coronavirus 2, Isolate USA-WA1/2020, NR-52281 (wild-type, WT). Virus isolates were passaged in Vero E6 cells (ATCC CRL-1586) as previously described⁶³. Virus concentrations were determined using plaque assays. 4×10⁵ A549-ACE2 cells were seeded in a flat 96-well plate overnight. On the second day, when the cell confluence reached ˜70%, A549-ACE2 cells were either preincubated with 5 nM or 10 nM ponatinib for 1 hour and washed. The pre-incubated viruses after the wash were used to infect A549-ACE2 cells at a multiplicity of infection (MOI) of 0.2. After infection, the medium containing virus was removed, and overlay medium containing FBS-free DMEM and 2% low-melting point agarose was added. At 72 hours post infection, infected cells were fixed by 4% paraformaldehyde (PFA) overnight and then stained with 0.2% crystal violet.

in vivo Infection Model

6-8-week-old K18-hACE2 mice were anesthetized with ketamine (80 mg/kg)/xylazine (8 mg/kg) and intranasally infected with 5×10³ PFU wild type SARS-CoV-2 in 25 11.1 DMEM, followed by intranasal treatment with PBS or 3 mg/kg ponatinib by oral gavage once daily. Infected mice were maintained in bio-containment unit isolator cages (Allentown, NJ, USA) in the USC ABLS3. Body weights of mice were monitored daily. Mice were euthanized using ketamine (100 mg/kg)/xylazine (10 mg/kg) when body weights dropped below 20% of their original body weights. RNA was isolated from lung to assess viral load using quantitative real-time PCR as described below. The lung of the infected mice was homogenized in PBS and the plaque assay was performed using Vero-E6 cells to detect the viral titer of the homogenized lung in PBS. The expression of SARS-CoV-2 viral protein NP was examined using immunohistochemistry (IHC) in the trachea, lung, and brain sections from infected mice as described below.

### Quantitative Real-Time PCR

Mouse lung tissues were homogenized in DMEM, and RNA was isolated using a PureLink RNA isolation kit (K156002, Invitrogen). Viral copy numbers were determined with the One-Step qPCR kit (1725150, BioRad).

### H&E and IHC

Tissues isolated from the experimental mice were placed in 10% neutral buffered formalin for a minimum of 72 hours. After paraffin embedding, 4-μm-thick sections were cut from the blocks. IHC with an anti-NP protein antibody (NB100-56576, Novus) as the primary antibody was performed. Stained slides were mounted and scanned for observation.

### REFERENCES

1. Hu, B., Guo, H., Zhou, P. & Shi, Z. L. Nat Rev Microbiol, doi:10.1038/s41579-020-00459-7 (2020). 2. Worldometer. Worldometer's COVID-19 data., <https://www.worldometers.info/coronayirus/country/us/>(2020). 3. Parums, D. V. Med Sci Monit 28, e935952, doi:10.12659/MSM.935952 (2022). 4. Tregoning, J. S., Flight, K. E., Higham, S. L., Wang, Z. & Pierce, B. F. Nat Rev Immunol 21, 626-636, doi:10.1038/s41577-021-00592-1 (2021). 5. Pia, L. & Rowland-Jones, S. Nat Rev Immunol, doi:10.1038/s41577-022-00681-9 (2022). 6. Yang, J., Surapaneni, M. & Schiffer, C. A. Expert Rev Hematol 15, 393-402, doi:10.1080/17474086.2022.2077187 (2022). 7. Ahmad, S., Johnson, G. L. & Scott, J. E. Biochem Biophys Res Commun 463, 888-893, doi:10.1016/j.bbrc.2015.06.029 (2015). 8. Gozgit, J. M. et al. Cancer Chemother Pharmacol 71, 1315-1323, doi:10.1007/s00280-013-2131-z (2013). 9. Tan, F. H., Putoczki, T. L., Stylli, S. S. & Luwor, R. B. Onco Targets Ther 12, 635-645, doi:10.2147/OTT.S189391 (2019).

What is claimed is:

1. A method of treating a coronavirus infection in a subject in need thereof, said method comprising administering to the subject a therapeutically effective amount of ponatinib.

2. The method of claim 1, wherein the coronavirus infection is a SARS-CoV-2 infection.

3. The method of claim 1, wherein the therapeutically effective amount is from 0.1 mg per day to 30 mg per day.

4. The method of claim 1, wherein the therapeutically effective amount is from 0.1 mg per day to 15 mg per day.

5. The method of claim 1, wherein the therapeutically effective amount is from 1 mg per day to 15 mg per day.

6. The method of claim 1, wherein the therapeutically effective amount is from 1 mg per day to 10 mg per day.

7. The method of claim 1, wherein ponatinib is administered daily from 1 to 21 days.

8. The method of claim 1, wherein ponatinib is administered daily from 1 to 14 days.

9. The method of claim 1, wherein ponatinib is administered daily from 1 to 10 days.

10. The method of claim 1, wherein ponatinib is administered daily from 1 to 7 days.

11. The method of claim 1, wherein ponatinib is administered daily from 3 to 10 days.

12. The method of claim 1, wherein ponatinib is administered daily from 3 to 7 days.

13. The method of claim 1, wherein ponatinib is administered orally.

14. The method of claim 1, wherein ponatinib is administered intravenously.

15. The method of claim 1, wherein ponatinib is administered intramuscularly.

16. The method of claim 1, further comprising administering to the subject a therapeutically effective amount of a second agent.

17. The method of claim 16, wherein the second agent is an antibody against a SARS-CoV-2 protein.

18. The method of claim 17, wherein the SARS-CoV-2-protein is a coronavirus spike protein.

19. The method of claim 17, wherein the SARS-CoV-2-protein is an ACE2 fusion protein.

20. The method of claim 16, wherein the second agent is Paxlovid or Veklury.
